(19) United States
(12) Patent Application Publication
(10) Pub. No.: US 2022/0031289 A1
(43) Pub. Date: Feb. 3, 2022

(54) ULTRASONIC PROBE

(71) Applicant: CANON MEDICAL SYSTEMS CORPORATION, Otawara-shi (JP)

(72) Inventors: Kentaro KIKUCHI, Saitama (JP); Takatoshi OKUMURA, Yaita (JP); Ryota OSUMI, Nasushiobara (JP); Takeshi SATO, Nasushiobara (JP)

(73) Assignee: CANON MEDICAL SYSTEMS CORPORATION, Otawara-shi (JP)

(21) Appl. No.: 17/443,848

(22) Filed: Jul. 28, 2021

(30) Foreign Application Priority Data
Jul. 29, 2020 (JP) 2020-128178

(51) Int. Cl. A61B 8/00

(52) U.S. Cl. CPC A61B 8/56; A61B 8/54; A61B 8/4444

(57) ABSTRACT

The ultrasonic probe according to any of embodiments includes a transmitting/receiving circuit, a power supply circuit, and a probe control circuit. The transmitting/receiving circuit is configured to control transmission and reception of ultrasonic waves. The power supply circuit is configured to function as a power source for the transmitting/receiving circuit. The probe control circuit is configured to determine a scan mode, and to control a frequency of switching noise of the power supply circuit according to the scan mode.

### CROSS-REFERENCE TO RELATED APPLICATION

This application is based upon and claims the benefit of priority from Japanese Patent Application No. 2020-128178, filed on Jul. 29, 2020, the entire contents of which are incorporated herein by reference.

### FIELD

Any of embodiments disclosed in specification and drawings relates to an ultrasonic probe.

### BACKGROUND

In the medical field, an ultrasonic diagnostic system is used for imaging the inside of a subject using ultrasonic waves generated by multiple transducers (piezoelectric elements) of an ultrasonic probe. The ultrasonic diagnostic system causes the ultrasonic probe, which is connected to the ultrasonic diagnostic system, to transmit ultrasonic waves into the subject, generates an echo signal based on a reflected wave, and acquires a desired ultrasonic image by image processing.

In the ultrasonic diagnostic system, miniaturized, lightweight, and wireless types have been developed in order to improve operability and maneuverability. Along with such developments, the number of electronic circuits in the ultrasonic probe tends to increase. Further, the ultrasonic probe may include a power supply unit having multiple switching regulators as a power source for each built-in electronic circuit. When a DC voltage is supplied from the power supply unit of the external ultrasonic diagnostic apparatus via the connector, the multiple switching regulators convert the DC voltage into an appropriate voltage, and supplies it to each electronic circuit within the ultrasonic probe.

### BRIEF DESCRIPTION OF THE DRAWINGS

FIG. 1 is a schematic diagram showing a configuration of an ultrasonic diagnostic system including an ultrasonic probe according to the first embodiment.

FIG. 2 is a schematic view showing a configuration of an ultrasonic diagnostic system including an ultrasonic probe according to a comparative example.

FIG. 3 is a graph showing a relationship between a transmission/reception band of the ultrasonic probe according to the comparative example and switching noise.

FIG. 4 is a graph showing a relationship between a transmission/reception band of the ultrasonic probe according to the first embodiment and switching noise.

FIG. 5 is a graph showing a relationship between a transmission/reception band of an ultrasonic probe according to a modified example and switching noise in a Doppler mode.

FIG. 6 is a graph showing a relationship between a transmission/reception band of the ultrasonic probe according to the modified example and switching noise in a B-mode.

FIG. 7 is a graph showing a relationship between a transmission/reception band of the ultrasonic probe according to the modified example and the switching noise in the Doppler mode.

FIG. 8 is a schematic view showing a configuration of an ultrasonic diagnostic system including an ultrasonic probe according to the second embodiment.

### DETAILED DESCRIPTION

An ultrasonic probe according to any of embodiments will be described with reference to the accompanying drawings.

The ultrasonic probe according to any of embodiments includes a transmitting/receiving circuit, a power supply circuit, and a probe control circuit. The transmitting/receiving circuit is configured to control transmission and reception of ultrasonic waves. The power supply circuit is configured to function as a power source for the transmitting/receiving circuit. The probe control circuit is configured to determine a scan mode, and to control a frequency of switching noise of the power supply circuit according to the scan mode.

### First Embodiment

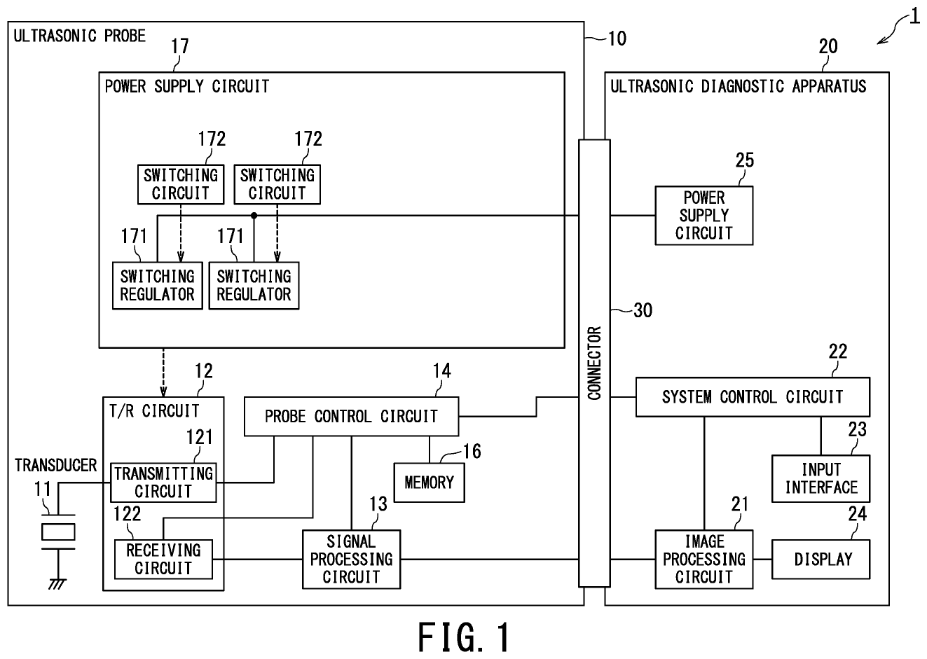

FIG. 1 is a schematic diagram showing a configuration of an ultrasonic diagnostic system including an ultrasonic probe according to the first embodiment.

FIG. 1 shows an ultrasonic diagnostic system 1 provided with an ultrasonic probe 10 according to the first embodiment. The ultrasonic diagnostic system 1 includes an ultrasonic probe 10, an ultrasonic diagnostic apparatus 20, and a connector 30. The ultrasonic probe 10 is connected to the ultrasonic diagnostic apparatus 20 via the connector (including a cable) 30.

The ultrasonic probe 10 includes multiple transducers (piezoelectric elements) 11, a transmitting/receiving (T/R) circuit 12, a signal processing circuit 13, a probe control circuit 14, a memory 16, and a power supply circuit 17. The circuits 12 and 13 are composed of an application specific integrated circuit (ASIC) or the like. However, the present invention is not limited to this case, and all or a part of the functions of the circuits 12 and 13 may be realized by the probe control circuit 14 executing a computer program.

The transducers 11 are provided on the front surface of the ultrasonic probe 10. The transducers 11 transmits and receives ultrasonic waves to and from a region covering a scan target. Each transducer is an electroacoustic conversion element, and has a function of converting an electric pulse into an ultrasonic pulse at the time of transmission and converting a reflected wave into an electric signal (received signal) at the time of reception.

The ultrasonic probe 10 is classified into types such as a linear type, a convex type, a sector type, etc. depending on differences in scanning system. Further, depending on the array arrangement dimension, the ultrasonic probe 10 is classified into a 1D array probe in which transducers are arrayed in a one-dimensional (1D) manner in the azimuth direction, and a 2D array probe in which transducers are arrayed in a two-dimensional (2D) manner in the azimuth direction and in the elevation direction. The 1D array probe includes a probe in which a small number of transducers are arranged in the elevation direction.

In the specification, when a three-dimensional (3D) scan, that is, a volume scan is executed, the 2D array probe having a scan type such as the linear type, the convex type, the sector type, or the like is used as the ultrasonic probe 10. Alternatively, when the volume scan is executed, the 1D probe having a scan type such as the linear type, the convex type, the sector type, and the like, and having a mechanism that mechanically oscillates in the elevation direction is used as the ultrasonic probe 10. The latter probe is also called a mechanical 4D probe.

The T/R circuit 12 has a transmitting circuit 121 and a receiving circuit 122. The T/R circuit 12 controls the transmission and reception of ultrasonic waves under the control of the probe control circuit 14. The T/R circuit 12 is an example of a transmitting/receiving unit.

The transmitting circuit 121 has a pulse generating circuit, a transmission delay circuit, a pulsar circuit and the like, and supplies a drive signal to ultrasonic transducers. The pulse generating circuit repeatedly generates rate pulses for forming transmission ultrasonic waves at a predetermined rate frequency. The transmission delay circuit converges the ultrasonic waves generated from the ultrasonic transducer of the ultrasonic probe 10 into a beam shape, and gives a delay time of each piezoelectric transducer necessary for determining the transmission directivity to each rate pulse generated by the pulse generating circuit. In addition, the pulsar circuit applies drive pulses to each ultrasonic transducer at a timing based on the rate pulses. The transmission delay circuit arbitrarily adjusts the transmission direction of the ultrasonic beam transmitted from a piezoelectric transducer surface by changing the delay time given to each rate pulse. The transmitting circuit 121 is an example of a transmitting unit.

The receiving circuit 122 has an amplifier circuit, an analog to digital (A/D) converter, an adder, and the like, receives the echo signal received by the ultrasonic transducers, and generate echo data by performing various processes on the echo signal. The amplifier circuit amplifies the echo signal for each channel, and performs gain correction processing. The A/D converter A/D-converts the gain-corrected echo signal, and gives a delay time necessary for determining the reception directivity to the digital data. The adder adds the echo signal processed by the A/D converter to generate echo data. By the addition processing of the adder, the reflection component from the direction corresponding to the reception directivity of the echo signal is emphasized. The receiving circuit 122 is an example of a receiving unit.

The signal processing circuit 13 includes a B-mode processing circuit (not shown) and a Doppler processing circuit (not shown).

Under the control of the probe control circuit 14, the B-mode processing circuit of the signal processing circuit 13 receives the echo data from the receiving circuit, performs logarithmic amplification, envelope detection processing and the like, thereby generate data (2D or 3D data) which signal intensity is represented by brightness of luminance. This data is an example of the raw data, and is generally called “B-mode data”.

The B-mode processing circuit may change the frequency band to be visualized by changing the detection frequency using filtering processing. By using the filtering processing function of the B-mode processing circuit, harmonic imaging such as the contrast harmonic imaging (CHI) or the tissue harmonic imaging (THI) is performed.

That is, the B-mode processing circuit may separate the reflected wave data of a subject into which the contrast agent is injected into harmonic data (or sub-frequency data) and fundamental wave data. The harmonic data (or sub-frequency data) refers to the reflected wave data of a harmonic component whose reflection source is the contrast agent (microbubbles or bubbles) in the subject. The fundamental wave data refers to the reflected wave data of a fundamental wave component whose reflection source is tissue in the subject. The B-mode processing circuit is able to generate B-mode data for generating contrast image data based on the reflected wave data (received signal) of the harmonic component, and to generate B-mode data for generating fundamental wave image data based on the reflected wave data (received signal) of the fundamental wave component.

In the THI using the filtering processing function of the B-mode processing circuit, it is possible to separate harmonic data or sub-frequency data which is reflected wave data (received signal) of a harmonic component from reflected wave data of the subject. Then, the B-mode processing circuit generates B-mode data for generating tissue image data in which the noise component is removed from the reflected wave data (received signal) of the harmonic component.

When the CHI or THI harmonic imaging is performed, the B-mode processing circuit may extract the harmonic component by a method different from the method using the above-described filtering. In harmonic imaging, an imaging method called the amplitude modulation (AM) method, the phase modulation (PM) method, or the AM-PM method in which the AM method and the PM method are combined is performed. In the AM method, the PM method, and the AM-PM method, ultrasonic transmission with different amplitudes and phases is performed multiple times on the same scanning line.

Thereby, the T/R circuit 12 generates and outputs multiple reflected wave data (received signal) in each scanning line. The B-mode processing circuit extracts harmonic components from the multiple reflected wave data (received signal) of each scanning line by performing addition/subtraction processing according to the modulation method. The B-mode processing circuit performs envelope detection processing etc. on the reflected wave data (received signal) of the harmonic component to generate B-mode data.

For example, when the PM method is performed, the T/R circuit 12 transmits the ultrasonic waves of the same amplitude and reversed-phase polarities, such as (−1, 1), twice by each scanning line under a scan sequence set by the probe control circuit 14. The T/R circuit 12 generates a reception signal based on transmission of “−1” and a reception signal based on transmission of “1”. The B-mode processing circuit adds these two reception signals. As a result, the fundamental wave component is removed, and a signal in which the second harmonic component mainly remains is generated. Then, the B-mode processing circuit performs envelope detection processing and the like on this signal to generate B-mode data using THI or CHI.

Alternatively, for example, in the THI, an imaging method using the second harmonic component and a difference tone component included in the received signal has been put to practical use. In the imaging method using the difference tone component, the transmission ultrasonic waves having, for example, a composite waveform combining a first fundamental waves with a center frequency “f1” and a second fundamental waves with a center frequency “f2” larger than the center frequency “f1” are transmitted from the ultrasonic probe 10. Such a composite waveform combines the first fundamental waves and a waveform with the second fundamental waves whose phases are adjusted with each other, such that the difference tone component having the same polarity as the second harmonic component is generated. The T/R circuit 12 transmits the transmission ultrasonic waves of the composite waveform, for example, twice while inverting the phase. In such a case, for example, the B-mode processing circuit removes the fundamental wave component by adding two received signals, and performs an envelope detection process etc. after extracting a harmonic component in which the difference tone component and the second harmonic component are mainly left.

Under the control of the probe control circuit 14, the Doppler processing circuit of the signal processing circuit 13 frequency-analyzes the phase information from the echo data from the receiving circuit, thereby generating data (2D or 3D data) by extracting multiple moving data of moving subject such as average speed, dispersion, power and the like. This data is an example of the raw data, and is generally called “Doppler data”. In the specification, the moving subject refers to, for example, blood flow, tissue such as heart wall, or contrast agent. The signal processing circuit 13 is an example of a signal processer.

The probe control circuit 14 includes processing circuitry (not shown) and a configuration memory (not shown). The probe control circuit 14 has a function of controlling the T/R circuit 12, the signal processing circuit 13 and the like to perform the scan according to the scan mode (e.g., M-mode, B-mode, and color mode), and a function of controlling the frequency of the switching noise of a switching regulator 171 of the power supply circuit 17. For example, the probe control circuit 14 controls the power supply circuit 17 so that the switching noise is out of the transmission/reception band of the ultrasonic probe 10 (upper part of FIG. 4). Alternatively, the probe control circuit 14 controls the power supply circuit 17 so that the signal to noise (S/N) deterioration degree due to switching noise is within the threshold within the transmission/reception band of the ultrasonic probe 10 (lower part of FIG. 4).

In such manner, the probe control circuit 14 is able to control the frequency of the switching noise by changing the switching frequency or changing the switching duty.

The processing circuitry of the probe control circuit 14 refers to an ASIC, a programmable logic device, etc. in addition to a dedicated or general purpose central processing unit (CPU), a micro processor unit (MPU), or a graphics processing unit (GPU). The programmable logic device may refer to, for example, a simple programmable logic device (SPLD), a complex programmable logic device (CPLD), a field programmable gate array (FPGA).

Further, the processing circuitry may be constituted by a single circuit or a combination of multiple independent circuit elements. In the latter case, the configuration memory may be provided individually for each circuit element, or a single memory may store programs corresponding to the functions of the multiple circuit elements.

The configuration memory of the probe control circuit 14 is constituted by a semiconductor memory element such as a random-access memory (RAM), a flash memory, a hard disk, an optical disk, or the like. The memory may be constituted by a portable medium such as a universal serial bus (USB) memory or a digital video disk (DVD). The probe control circuit 14 is an example of a probe controller.

The memory 16 stores transmission/reception band information of each ultrasonic probe in advance.

The power supply circuit 17 includes a switching regulator 171 and a switching circuit 172. The power supply circuit 17 functions as a power source for electronic circuits such as the T/R circuit 12. The switching regulator 171 is a type of DC-DC converter that converts a direct current (DC) voltage into a direct current voltage having a different value and outputs the voltage. The probe control circuit 14 reads transmission/reception band information of each ultrasonic probe from the memory 16 and controls the switching circuit 172 based on the information. Thereby, the probe control circuit 14 is able to set the switching regulator 171 to a desired output voltage. The power supply circuit 17 is an example of a power supply unit.

The ultrasonic diagnostic apparatus 20 includes an image processing circuit 21, a system control circuit 22, an input interface 23, a display 24, and a power supply circuit 25. The ultrasonic diagnostic apparatus may be configured to include both the input interface 23 and the display 24, or to include only one of the input interface 23 and the display 24. In the following description, a case where the ultrasonic diagnostic apparatus 20 includes both the input interface 23 and the display 24 will be described.

Under the control of the system control circuit 22, the image processing circuit 21 generates an ultrasonic image represented in a predetermined luminance range as image data based on the received signal received by the ultrasonic probe 10. For example, the image processing circuit 21 generates, as the ultrasonic image, a B-mode image in which the intensity of the reflected wave is represented by brightness from the 2D B-mode data generated by the B-mode processing circuit of the signal processing circuit 13. Further, the image processing circuit 21 generates, as the ultrasonic image, a color Doppler image which may be an average velocity image, a distributed image, a power image, or a combination image thereof that represents moving object information from the 2D Doppler data generated by the Doppler processing circuit of the signal processing circuit 13.

In the specification, the image processing circuit 21 generally converts (scan-converts) a scanning line signal string of ultrasonic scanning into a scanning line signal string of a video format typified by a television or the like, thereby generating ultrasonic image data for display. Specifically, the image processing circuit 21 performs coordinate conversion according to the scanning form of ultrasonic waves by the ultrasonic probe 10, thereby generating ultrasonic image data for display. In addition to scan conversion, the image processing circuit 21 also performs various image processing such as image processing that regenerates an average brightness image using multiple image frames after the scan conversion processing (smoothing processing), and image processing that uses a differential filter in the image (edge enhancement processing). Further, the image processing circuit 21 synthesizes character information, scales, body marks, and the like of various parameters with the ultrasonic image data.

That is, the B-mode data and the Doppler data are ultrasonic image data before the scan conversion processing. The data generated by the image processing circuit 21 is ultrasonic image data for display after the scan conversion processing. The B-mode data and Doppler data are also referred to as “raw data”. The image processing circuit 21 generates 2D ultrasonic image data for display from the 2D ultrasonic image data before the scan conversion processing.

Further, the image processing circuit 21 includes a graphics processing unit (GPU), a video RAM (VRAM), and the like. The image processing circuit 21, under the control of the system control circuit 22, adjusts the signal strength of the ultrasonic image (e.g., a live image) that the system control circuit 22 requests to display, and displays such image on the display 24. The image processing circuit 21 is an example of an image processor.

Since the system control circuit 22 has the same configuration as the probe control circuit 14 of the ultrasonic probe 10, the description thereof will be omitted. Further, the memory of the system control circuit 22 stores various processing programs (including an operating system (OS) and the like in addition to the application program) used in the processing circuitry of the system control circuit 22 and data necessary for executing the program. In addition, a graphical user interface (GUI) can be included in the OS. The GUI is for displaying information on the display 24 to the operator by using a lot of graphics and performing basic operations by the input interface 23. The system control circuit 22 is an example of a system controller.

The input interface 23 includes an input device operable by an operator, and a circuit for inputting a signal from the input device. The input device may be a trackball, a switch, a mouse, a keyboard, a touch pad for performing an input operation by touching an operation surface, a touch screen in which a display screen and a touch pad are integrated, a non-contact input circuit using an optical sensor, an audio input circuit, and the like. When the input device is operated by the operator, the input interface 23 generates an input signal corresponding to the operation and outputs it to the system control circuit 22. The input interface 23 is an example of an input unit.

The display 24 is constituted by a general display output device such as a liquid crystal display or an organic light emitting diode (OLED) display. The display 24 displays various kinds of information under the control of the system control circuit 22. The display 24 is an example of a display unit.

The power supply circuit 25 includes a transformer, a rectifier circuit, a smoothing circuit, and the like. The transformer converts the voltage of the commercial power supply source (alternating current) using the transformer. The rectifier circuit converts alternating current (AC) voltage to direct current (DC) voltage. The smoothing circuit rectifies the fluctuation of the voltage to acquire a DC voltage having a stable voltage. The power supply circuit 25 supplies a DC voltage to the electronic circuit in the ultrasonic diagnostic apparatus 20 and also supplies the DC voltage to the power supply circuit 17 of the ultrasonic probe 10 via the connector 30. The power supply circuit 25 is an example of a power supply unit.

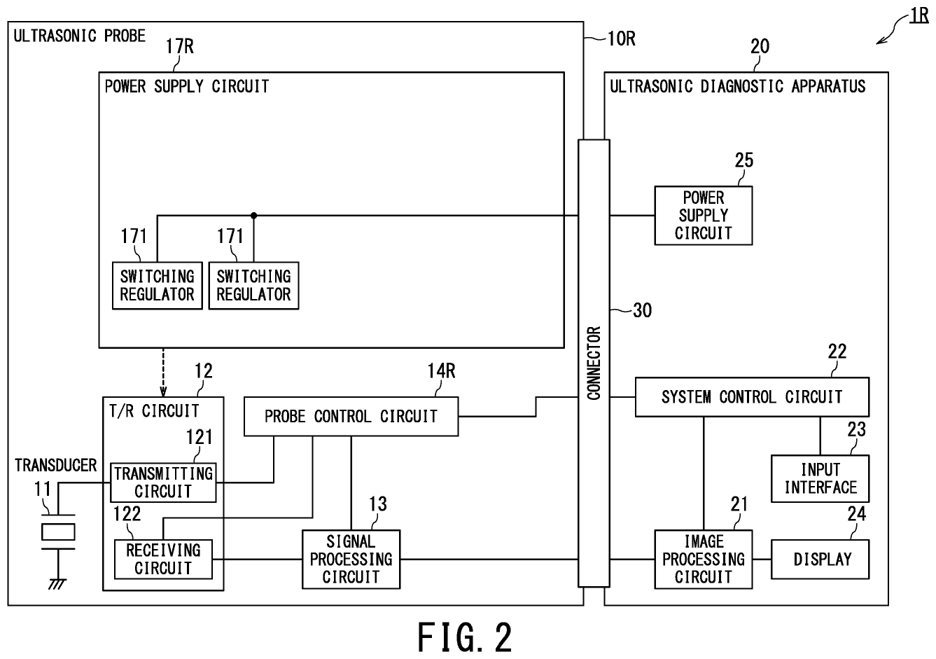

FIG. 2 is a schematic view showing a configuration of an ultrasonic diagnostic system including an ultrasonic probe according to a comparative example.

FIG. 2 shows an ultrasonic diagnostic system 1R provided with an ultrasonic probe 10R according to a comparative example. The ultrasonic diagnostic system 1R includes an ultrasonic probe 10R, an ultrasonic diagnostic apparatus 20, and a connector 30. The ultrasonic probe 10R is connected to the ultrasonic diagnostic apparatus 20 via the connector (including a cable) 30.

In the ultrasonic diagnostic system 1R shown in FIG. 2, the same members as those of the ultrasonic diagnostic system 1 shown in FIG. 1 are designated with the same reference numerals, and the description thereof will be omitted.

The ultrasonic probe 10R includes multiple transducers (piezoelectric elements) 11, a T/R circuit 12, a signal processing circuit 13, a probe control circuit 14R, and a power supply circuit 17R.

The probe control circuit 14R includes processing circuitry (not shown) and a memory (not shown). Unlike the probe control circuit 14 (shown in FIG. 1), the probe control circuit 14R has only a function of controlling the T/R circuit 12, the signal processing circuit 13, and the like to perform scanning according to the scan mode so as to perform the scan.

The power supply circuit 17R has multiple switching regulators 171. The DC voltage is supplied to the switching regulators 171 from the power supply circuit 25 of the ultrasonic diagnostic apparatus 20 via the connector 30. The switching regulators 171 convert the DC voltage into an appropriate voltage, and supply the converted voltage to each electronic circuit in the ultrasonic probe 10R.

Generally, there are linear regulators and switching regulators, and the switching regulators are used because of the small size, light weight, and high efficiency. The switching regulators generate switching noise. The switching frequency of a switching regulator is generally several tens of kHz to several MHz, and such frequency and harmonics result in switching noise. On the other hand, the transmission/reception band of ultrasonic waves is several MHz. Therefore, when the switching noise gets into the T/R circuit 12 in the ultrasonic probe 10R, the switching noise affects the analog transmission/reception signal, and lower the quality of the ultrasonic images.

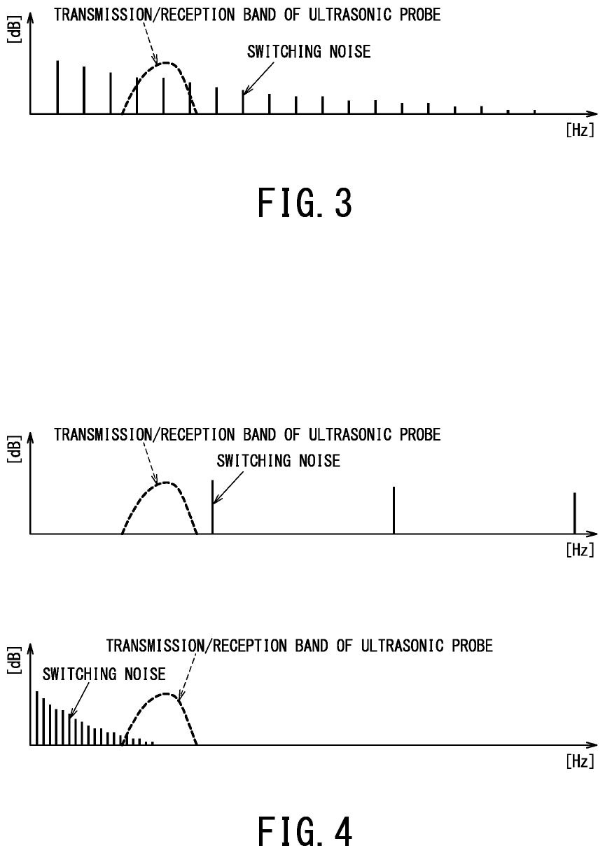

FIG. 3 is a graph showing a relationship between the transmission/reception band of the ultrasonic probe 10R and the switching noise.

As shown in FIG. 3, the switching noise of the switching regulator 171 of the ultrasonic probe 10R is several tens of kHz to several MHz. On the other hand, since the ultrasonic transmission/reception band is several MHz, the switching noise and the ultrasonic wave transmission/reception band overlap in the same band. Therefore, the switching noise affects the analog transmission/reception signal.

As a countermeasure against the switching noise, there is a method of removing noise by using a noise filter element. In that case, it is necessary to secure a region for mounting the noise filter element in the ultrasonic probe 10R. There is also a method of relatively reducing the influence of switching noise by widening the dynamic range. In that case, the small-amplitude echo is buried in noise.

Assume that the ultrasonic probe 10 is configure in the manner as shown in FIG. 1. The probe control circuit 14 shown in FIG. 1 acquires transmission/reception band information of the ultrasonic probe 10 from the transmission/reception band information of each ultrasonic probe stored in the memory 16. Then, the probe control circuit 14 controls the frequency of the switching noise by changing the switching frequency or changing the switching duty.

FIG. 4 is a graph showing the relationship between the transmission/reception band of the ultrasonic probe 10 and the switching noise. The upper part of FIG. 4 shows a case where the switching noise is controlled so as to be out of the transmission/reception band of the ultrasonic probe 10. The lower part of FIG. 4 shows a case where control is performed so that an S/N deterioration degree due to the switching noise is within a threshold within the transmission/reception band of the ultrasonic probe 10.

As shown in the upper part of FIG. 4, the probe control circuit 14 controls the switching noise of the switching regulator 171 of the ultrasonic probe 10 so as to be in a band different from the ultrasonic transmission/reception band. In this case, the primary component of the switching noise appears on the high frequency side of the transmission/reception band of the ultrasonic probe 10. Further, as shown in the lower part of FIG. 4, the probe control circuit 14 controls so that the S/N deterioration degree due to the switching noise of the switching regulator 171 of the ultrasonic probe 10 is within the threshold within the transmission/reception band of the ultrasonic probe 10. In that case, the primary component appears on the low frequency side of the transmission/reception band of the ultrasonic probe 10 in consideration of the attenuation of each switching noise appearing in multiple frequencies.

As described above, according to the ultrasonic probe 10, the switching noise of the switching regulator 171 is able to be reduced to zero or minimized within the transmission/reception band of the ultrasonic probe 10, and the influence on the images due to the switching noise caused by the switching regulator 171 of the power supply circuit 17 can be suppressed.

### Modified Example

The transmission/reception band of the ultrasonic probe 10 differs depending on a scan mode set among the multiple scan modes. Therefore, the probe control circuit 14 may control the frequency of the switching noise by changing the switching frequency or changing the switching duty according to the set scan mode.

In addition to the function of executing the scan, the probe control circuit 14 determines the scan mode, and controls the frequency of the switching noise of the switching regulator 171 of the power supply circuit 17 according to the scan mode.

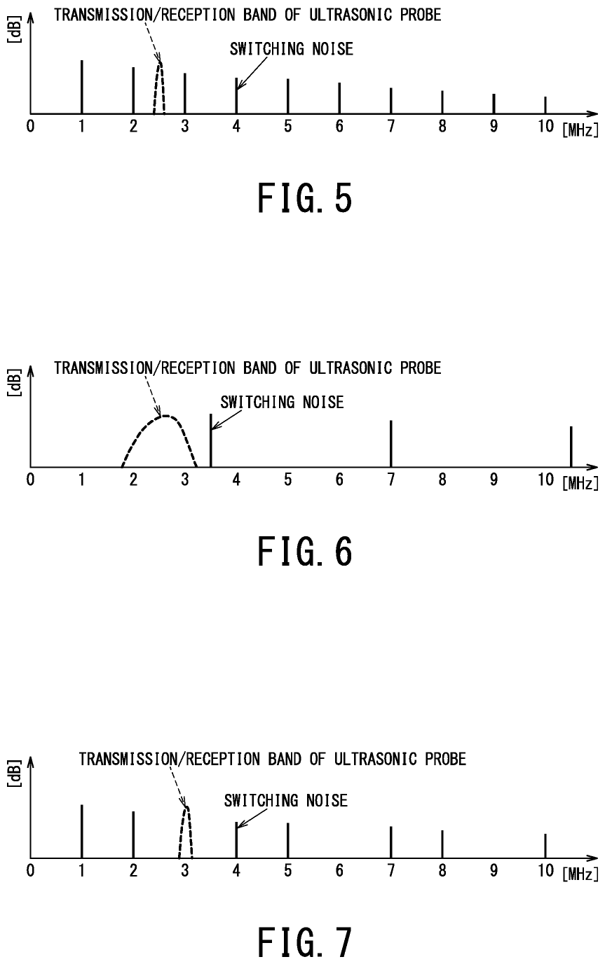

FIG. 5 is a graph showing a relationship between the transmission/reception band of the ultrasonic probe 10 and the switching noise in the Doppler mode.

In the Doppler mode with a transmission frequency of 2.5 [MHz], the received signal band is 2.5 [MHz]±0.05 [MHz], as shown in FIG. 5. In order to control the switching noise so as to avoid this band, the probe control circuit 14 controls the switching frequency to be 1 [MHz] such that the frequency of the switching noise becomes 1 [MHz], 2 [MHz], 3 [MHz], . . . . Thereby, in the Doppler mode, it is possible to set the switching noise while avoiding the transmission/reception band of the ultrasonic probe 10.

FIG. 6 is a graph showing a relationship between the transmission/reception band of the ultrasonic probe 10 and the switching noise in the B-mode.

In the case of the B-mode having a transmission frequency of 2.5 [MHz], as shown in FIG. 6, the received signal band is 1.75 [MHz] to 3.25 [MHz] (center frequency 2.5 [MHz]±0.75 [MHz]). In order to control the switching noise so as to avoid this band, the probe control circuit 14 controls the switching frequency to be 3.5 [MHz] such that the frequency of the switching noise becomes 3.5 [MHz], 7.0 [MHz], 10.5 [MHz], . . . . Thereby, in the B-mode, it is possible to set the switching noise while avoiding the transmission/reception band of the ultrasonic probe 10.

FIG. 7 is a graph showing a relationship between the transmission/reception band of the ultrasonic probe 10 and the switching noise in the Doppler mode.

In the case of Doppler with a transmission frequency of 3.0 [MHz], the received signal band is 3.0 [MHz]±0.05 [MHz], as shown in FIG. 7. In order to control the switching noise so as to avoid this band, the probe control circuit 14 reduces the duty ratio to 1/3 when the switching frequency of the switching regulator 171 is 1 [MHz]. In such manner, the switching noise of 3×n [MHz] (“n” is a positive integer) can be reduced to zero. Thereby, in the Doppler mode, it is possible to avoid the switching noise getting into the transmission/reception band of the ultrasonic probe 10.

### Second Embodiment

The wireless ultrasonic probe is used with the battery in the ultrasonic probe being fully charged. When the battery in the ultrasonic probe is not sufficiently charged, power to the battery will be supplied by connecting the external power source with the power supply circuit of the wireless ultrasonic probe by wire while using the ultrasonic probe. Therefore, switching noise caused by a battery charging circuit of the ultrasonic probe may get into each electronic circuit in the ultrasonic probe.

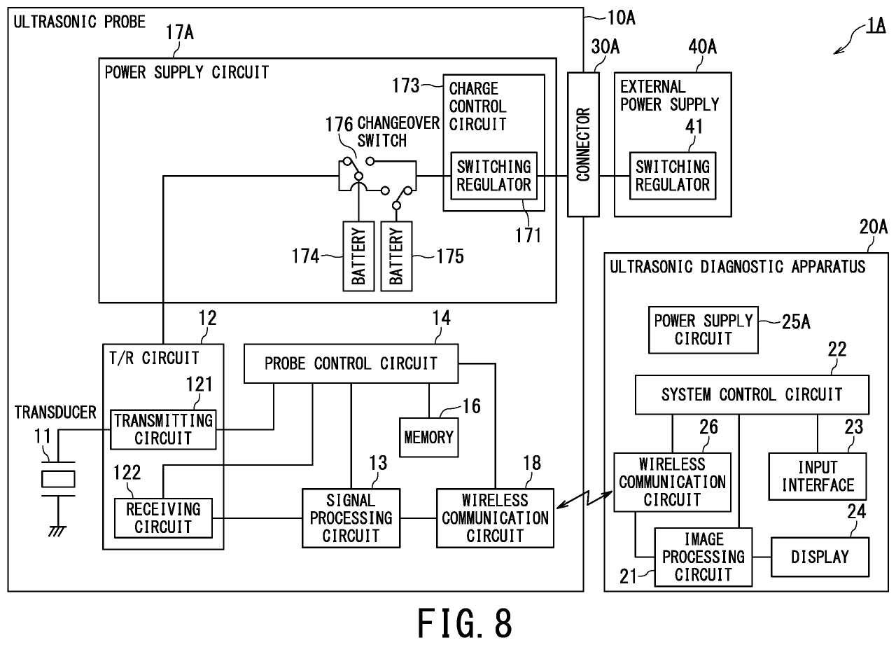

FIG. 8 is a schematic view showing a configuration of an ultrasonic diagnostic system including an ultrasonic probe according to the second embodiment.

FIG. 8 shows an ultrasonic diagnostic system 1A including an ultrasonic probe 10A according to the second embodiment. The ultrasonic diagnostic system 1A includes an ultrasonic probe 10A, an ultrasonic diagnostic apparatus 20A, and a connector 30A. The ultrasonic probe 10A is connected to the ultrasonic diagnostic apparatus 20A via the connector (including a cable) 30A.

The ultrasonic probe 10A includes multiple transducers (piezoelectric elements) 11, a T/R circuit 12, a signal processing circuit 13, a probe control circuit 14, a memory 16, a power supply circuit 17A, and a wireless communication circuit 18. The circuits 12 to 13 are configured by an ASIC or the like. However, the present invention is not limited to this case, and all or a part of the functions of the circuits 12 to 13 may be realized by the probe control circuit 14 executing a computer program.

The power supply circuit 17A includes a charge control circuit 173, multiple batteries 174 and 175, and a changeover switch 176. The battery of the ultrasonic probe 10A is divided into two. Then, the probe control circuit 14 switches a power supply source of the T/R circuit 12 in the ultrasonic probe 10A using the batteries 174 and 175. The probe control circuit 14 uses one battery as the power source to supply the T/R circuit 12, and connects the other battery to the charging side. In such manner, the T/R circuit 12 in the ultrasonic probe 10A is separated from the charge control circuit 173 and an external power source 40A, while acquiring power from the battery. Therefore, the switching noise caused by the charge control circuit 173 and the power supply circuit 17A will not get into the T/R circuit 12.

The ultrasonic diagnostic apparatus 20A includes an image processing circuit 21, a system control circuit 22, an input interface 23, a display 24, a power supply circuit 25A, and a wireless communication circuit 26. In some cases, the input interface 23 and the display 24 are provided to form an ultrasonic diagnostic apparatus. The ultrasonic diagnostic apparatus may be configured with only one of the input interface 23 and the display 24. In the following description, a case where the ultrasonic diagnostic apparatus 20 includes both the input interface 23 and the display 24 will be described.

The power supply circuit 25A includes a transformer, a rectifier circuit, a smoothing circuit, and the like. The transformer converts the voltage of the commercial power supply source (alternating current). The rectifier circuit converts alternating current (AC) voltage to direct current (DC) voltage. The smoothing circuit rectifies the fluctuation of the voltage to acquire a stable DC voltage. The power supply circuit 25A supplies a DC voltage to the electronic circuit in the ultrasonic diagnostic apparatus 20. The power supply circuit 25A is an example of a power supply unit.

In FIG. 8, the same parts as those of the ultrasonic diagnostic system 1 shown in FIG. 1 are designated with the same reference numerals, and the description thereof will be omitted.

The probe control circuit 14 is able to switch the power supply source of the T/R circuit 12 in the ultrasonic probe 10A using the batteries 174 and 175, thereby the circuit 14 is able to control switching noise caused by the battery charging circuit. The above-mentioned modified example may also be applied to the ultrasonic probe 10A according to the second embodiment.

As described above, according to the ultrasonic probe 10A, it is possible to suppress the influence on the images due to the switching noise caused by the battery charging circuit.

According to at least one embodiment described above, it is possible to suppress the influence of switching noise on the image.

While certain embodiments have been described, these embodiments have been presented by way of example only, and are not intended to limit the scope of the inventions. Indeed, the novel methods and systems described herein may be embodied in a variety of other forms; furthermore, various omissions, substitutions, changes, and combinations of embodiments in the form of the methods and systems described herein may be made without departing from the spirit of the inventions. The accompanying claims and their equivalents are intended to cover such forms or modifications as would fall within the scope and spirit of the inventions.

What is claimed is:

1. An ultrasonic probe comprising:
a transmitting/receiving circuit configured to control transmission and reception of ultrasonic waves;
a power supply circuit configured to function as a power source for the transmitting/receiving circuit; and
a probe control circuit configured to determine a scan mode, and to control a frequency of switching noise of the power supply circuit according to the scan mode.

2. The ultrasonic probe according to claim 1, wherein
the power supply circuit includes a switching regulator and a switching circuit, and
the probe control circuit is configured to control the frequency of switching noise of the switching regulator.

3. The ultrasonic probe according to claim 2, wherein
the probe control circuit is configured to control the power supply circuit such that the switching noise is out of a transmission/reception band of the ultrasonic probe.

4. The ultrasonic probe according to claim 2, wherein
the probe control circuit is configured to control the power supply circuit such that a signal to noise (S/N) deterioration degree of the switching noise is within a threshold within a transmission/reception band of the ultrasonic probe.

5. The ultrasonic probe according to claim 2, wherein
the probe control circuit is configured to control the switching noise by changing a switching frequency of the switching circuit.

6. The ultrasonic probe according to claim 2, wherein
the probe control circuit is configured to control the switching noise by changing a switching duty of the switching circuit.

7. The ultrasonic probe according to claim 1, wherein
the power supply circuit includes multiple batteries, and
the probe control circuit is configured to switch a power supply source of the transmitting/receiving circuit using multiple batteries.

8. An ultrasonic probe comprising:
a transmitting/receiving circuit configured to control transmission and reception of ultrasonic waves;
a power supply circuit including a switching regulator and a switching circuit, and configured to function as a power source for the transmitting/receiving circuit; and
a probe control circuit configured to control the power supply circuit such that an S/N deterioration degree of switching noise of the switching regulator is within a threshold within a transmission/reception band of the ultrasonic probe.
